(19) United States
(12) Patent Application Publication
(10) Pub. No.: US 2018/0137235 A1
(43) Pub. Date: May 17, 2018

(54) SYSTEM AND METHOD FOR PROCESSING GENOTYPE INFORMATION RELATING TO OPIOID RISK

(71) Applicant: PROOVE BIOSCIENCES, INC., Irvine, CA (US)

(72) Inventor: Brian MESHKIN, Ladera Ranch, CA (US)

(21) Appl. No.: 15/570,306

(22) PCT Filed: Apr. 28, 2016
(86) PCT No.: PCT/US16/29793

§ 371 Date: Oct. 27, 2017

Related U.S. Application Data
(60) Provisional application No. 62/153,733, filed on Apr. 28, 2015.

(51) Int. Cl. G06F 19/18; G06F 19/22; G16H 50/30; G16H 10/60

(52) U.S. Cl. CPC G06F 19/18; G16H 10/60; G16H 50/30; G06F 19/22

(57) ABSTRACT

There are methods, apparati and computer readable mediums associated with determining patient information, including DNA information, and determining from the DNA information whether a subject genotype of a human subject includes one or more SNP diploid polymorphisms by detecting, utilizing a detection technology and the DNA information, a presence or absence of the one or more SNP diploid polymorphisms in the subject genotype. The SNP diploid polymorphisms are selected from a SNP diploid group and utilized in determining an opioid dependency risk associated with the human subject.

### PRIORITY

This application claims priority to U.S. Provisional Application No. 62/153,733 entitled “System and Method for Processing Genotype Information Relating to Opioid Risk” by Brian Meshkin filed on Apr. 28, 2015, which is incorporated herein by reference in its entirety.

### CROSS-REFERENCE TO RELATED APPLICATIONS

This application is related to pending U.S. Utility application Ser. No. 13/924,540 entitled “System and Method for Processing Genotype Information Relating to Treatment with Pain Medication” by Brian Meshkin filed on Jun. 22, 2013, which is incorporated herein by reference in its entirety.

### BACKGROUND

In nature, organisms of the same species usually differ from each other in various aspects such as in their appearance or in one or more aspects of their biology. The differences are often based on genetic distinctions, some of which are called polymorphisms. Polymorphisms are often observed at the level of the whole individual (i.e., phenotype polymorphism), in variant forms of proteins and blood group substances (i.e., biochemical polymorphism), morphological features of chromosomes (i.e., chromosomal polymorphism) or, at the level of DNA, in differences of nucleotides and/or nucleotide sequences (i.e., genetic polymorphism).

Examples of genetic polymorphisms include alleles and haplotypes. An allele is an alternative form of a gene, such as one member of a pair, that is located at a specific position on a chromosome. A haplotype is a combination of alleles, or a combination of single nucleotide polymorphisms (SNPs) on the same chromosome. An example of a genetic polymorphism is an occurrence of one or more genetically alternative phenotypes in a subject due to the presence or absence of an allele or haplotype.

Genetic polymorphisms can play a role in determining differences in an individual's response to a species of drug, a drug dosage or a therapy including one drug or a combination of drugs. Pharmacogenetics and pharmacogenomics are multidisciplinary research efforts to study the relationships among genotypes, gene expression profiles, and phenotypes, as often expressed through the variability between individuals in response to drugs taken. Since the initial sequencing of the human genome, more than a million SNPs have been identified. Some of these SNPs have been used to predict clinical predispositions or responses based upon data gathered from pharmacogenomic studies.

Chronic pain affects up to 100 million Americans (more than heart disease, cancer, and diabetes combined) and has clinical and public health implications. Opioids are effective for pain control but may cause serious adverse effects including addiction. Addiction is a chronic behavioral pattern of drug dependence and abuse associated with an underlying neurobiological dysfunction is a common outcome from opioid treatment. In recent years, the consumption of opioid medication has increased 300%, and deaths associated with opioid poisoning more than tripled. Clearly, there is a need to reliably identify, prevent, and treat opioid addiction in people with pain. However, an optimal use of opioids must include comprehensive evaluation of risk associated with potential addiction and misuse of narcotic pain killers. Currently available screening tools validated for the assessment of potential opioid addiction such as Opioid Risk Tool (ORT) and the Screener and Opioid Assessment for Pain Patients (SOAPP®) are based on patient' self-report of behavior, personal and family medical history, and environmental factors. Like other self-reported outcomes, these data may suffer from trust issues and memory bias and are hard to verify. Regardless of the accuracy of the data provided via these screening tools, physicians are able to predict the development of opioid dependence (OD) and abuse with only a 50% chance of success. Most patients who are candidates for or taking prescription opioid pain medications do not control their pain well with medications, so doctors often increase dose, potency, or class of pain medications. Opioid abuse is one of the largest factors in increased healthcare costs for workers' compensation cases. The cost of chronic pain treatment often outpaces cancer and heart disease—combined. Healthcare payers often pay for both the drug being abused and the treatment of the abuse—both ways.

In pharmacogenomics, there is a desire to identify new polymorphisms and haplotypes associated with opioid substance abuse risk in patients who are candidates for or taking prescription opioid pain medications. The genotype information of a patient may help a prescriber understand whether the patient is at risk for opiod substance abuse.

A patient's genotype information is often utilized to help a prescriber decide between medications based on information associated with a patient's genetic profile (i.e., genotype information). There is a desire to utilize a patient's genotype information in determining the patient's predisposition to opioid substance abuse risk. There is also a desire for methods for predicting and/or diagnosing individuals exhibiting irregular predispositions to opioid substance abuse risk. Furthermore, there is also a desire to determine genetic information, such as polymorphisms, which may be utilized for predicting variations in opioid substance abuse risk among individuals. There is also a desire to implement systems processing and distributing the detected genetic information in a systematic way. Such genetic information would be useful in providing prognostic information about treatment options for a patient.

Although it is known generally that opioid substance abuse may be associated with genetics—a factor not routinely considered, there is no rigourous methodology to systematically provide doctor's with an ability to identify patients who may misuse narcotics and/or have a genetic predisposition for risk of abuse. Such systems and methods would be beneficial to provide information that improves accuracy in identifying patients at risk for opioid substance abuse.

Given the foregoing, and to address the above-described limitations, systems and methods are desired for identifying, estimating and/or determining a potential for success of an individual patient's clinical outcome in response to being prescribed an opioid pain medication.

### SUMMARY

This summary is provided to introduce a selection of concepts that are further described in the Detailed Description below. The genes, polymorphisms, sequences and sequence identifiers (i.e., SEQ IDs or SEQ ID Numbers) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with a predisposition to opioid substance abuse risk in a patient. The prognostic information is derived from genotype information about a patient's gene profile. The genotype information may be obtained by, inter alia, assaying a sample of genetic material associated with a patient.

The systems, methods and CRMs, according to the principles of the invention, can be utilized to determine prognostic information associated with opioid substance abuse risk based on the patient's opioid risk predisposition (ORP). The prognostic information may be used for addressing prescription needs or determining therapy directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with pain perception predisposition in a patient, in accordance with the principles of the invention.

According to a first principle of the invention, there is a method. The method may include facilitating a processing of and/or processing (1) data and/or (2) information and/or (3) at least one signal, the (1) data and/or (2) information and/or (3) at least one signal based, at least in part, on any combination of at least part of the the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group consisting of DRD1-ANC, DRD1-HET, and DRD1-NONA in the DRD1 gene, COMT(2)-ANC, COMT(2)-HET, and COMT(2)-NONA in the COMT gene, SLC6A3-ANC, SLC6A3-HET, and SLC6A3-NONA in the SLC6A3 gene, SLC6A4-ANC, SLC6A4-HET, and SLC6A4-NONA in the SLC6A4 gene, GABRG2-ANC, GABRG2-HET, and GABRG2-NONA in the GABRG2 gene, OPRK1-ANC, OPRK1-HET, and OPRK1-NONA in the OPRK1 gene, DBH-ANC, DBH-HET, and DBH-NONA in the DBH gene, OPRM1-ANC, OPRM1-HET, and OPRM1-NONA in the OPRM1 gene, DRD2(ANKK1)-ANC, DRD2(ANKK1)-HET, and DRD2(ANKK1)-NONA in the DRD2 gene, MTHFR-ANC, MTHFR-HET, and MTHFR-NONA in the MTHFR gene, DRD4-ANC, DRD4-HET, and DRD4-NONA in the DRD4 gene, and HTR2A-ANC, HTR2A-HET, and HTR2A-NONA in the HTR2A gene; and determining an opioid dependency risk associated with the human subject based, at least in part, on the presence or absence of the one or more SNP diploid polymorphisms in the subject genotype.

The method may also include wherein the (1) data and/or (2) information and/or (3) at least one signal are further based, at least in part, on any combination of the following: determining a comparing of a region, including the one or more SNP diploid polymorphisms, of the subject genotype with a corresponding region of a predetermined reference genotype, wherein characteristics of the corresponding region of the reference genotype are based upon a predetermined population norm; determining prognostic information associated with the human subject based on the determined opioid dependency risk; and determining a therapy for the human subject based on the determined prognostic information associated with the human subject, wherein the method for determining the opioid dependency risk associated with the human subject, is an ex vivo method; determining demographic information associated with the human subject as part of the patient information; and determining from the demographic information whether the human subject is characterized as being associated with one or more demographic phenotypes, wherein the determining of the opioid dependency risk associated with the human subject is based, at least in part, on the presence or absence of the one or more demographic phenotypes in the patient information associated with the human subject, wherein the one or more demographic phenotypes associated with the human subject are selected from a factor associated with the age of the human subject, a history of a mental health disorder other than depression in the human subject, a history of depression in the human subject, a history of alcoholism in the human subject, a history of illegal drug use by the human subject, a history of legal drug abuse by the human subject, or a combination thereof. The one or more SNP diploid polymorphisms may include at least any number of two through twelve SNP diploid polymorphisms from the SNP diploid group.

According to a second principle of the invention, there is an apparatus. The apparatus may include any combination of at least one processor; and at least one memory including computer program code for one or more programs, the at least one memory and the computer program code configured to, with the at least one processor, cause the apparatus to perform at least the following, determine patient information, including DNA information, associated with a human subject; determine from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group consisting of DRD1-ANC, DRD1-HET, and DRD1-NONA in the DRD1 gene, COMT(2)-ANC, COMT(2)-HET, and COMT(2)-NONA in the COMT gene, SLC6A3-ANC, SLC6A3-HET, and SLC6A3-NONA in the SLC6A3 gene, SLC6A4-ANC, SLC6A4-HET, and SLC6A4-NONA in the SLC6A4 gene, GABRG2-ANC, GABRG2-HET, and GABRG2-NONA in the GABRG2 gene, OPRK1-ANC, OPRK1-HET, and OPRK1-NONA in the OPRK1 gene, DBH-ANC, DBH-HET, and DBH-NONA in the DBH gene, OPRM1-ANC, OPRM1-HET, and OPRM1-NONA in the OPRM1 gene, DRD2(ANKK1)-ANC, DRD2(ANKK1)-HET, and DRD2(ANKK1)-NONA in the DRD2 gene, MTHFR-ANC, MTHFR-HET, and MTHFR-NONA in the MTHFR gene, DRD4-ANC, DRD4-HET, and DRD4-NONA in the DRD4 gene, and HTR2A-ANC, HTR2A-HET, and HTR2A-NONA in the HTR2A gene; and determine an opioid dependency risk associated with the human subject based, at least in part, on the presence or absence of the one or more SNP diploid polymorphisms in the subject genotype.

According to a third principle of the invention, there is a non-transitory computer readable medium. The medium may store any combination of computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on any combination of at least part of the the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group consisting of DRD1-ANC, DRD1-HET, and DRD1-NONA in the DRD1 gene, COMT(2)-ANC, COMT(2)-HET, and COMT(2)-NONA in the COMT gene, SLC6A3-ANC, SLC6A3-HET, and SLC6A3-NONA in the SLC6A3 gene, SLC6A4-ANC, SLC6A4-HET, and SLC6A4-NONA in the SLC6A4 gene, GABRG2-ANC, GABRG2-HET, and GABRG2-NONA in the GABRG2 gene, OPRK1-ANC, OPRK1-HET, and OPRK1-NONA in the OPRK1 gene, DBH-ANC, DBH-HET, and DBH-NONA in the DBH gene, OPRM1-ANC, OPRM1-HET, and OPRM1-NONA in the OPRM1 gene, DRD2(ANKK1)-ANC, DRD2(ANKK1)-HET, and DRD2(ANKK1)-NONA in the DRD2 gene, MTHFR-ANC, MTHFR-HET, and MTHFR-NONA in the MTHFR gene, DRD4-ANC, DRD4-HET, and DRD4-NONA in the DRD4 gene, and HTR2A-ANC, HTR2A-HET, and HTR2A-NONA in the HTR2A gene; and determining an opioid dependency risk associated with the human subject based, at least in part, on the presence or absence of the one or more SNP diploid polymorphisms in the subject genotype.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genotype and phenotype information to identify their genetic predisposition to opioid substance abuse risk. The genetic predisposition may be associated with the selection of an opioid pain medication, a dosage of the opioid pain medication and the utilization of the opioid pain medication in a regimen for treating the patient's medical condition.

The prognostic information may also be utilized for determining dose adjustments that may help a prescriber understand why a patient is or is not responding to an opioid medication dosage, such as an “average” dose. The prognostic information may also be utilized by a prescriber to decide between medications based on the patient's genetic predisposition to opioid substance abuse risk. The prognostic information may also be utilized for predicting and/or diagnosing individuals exhibiting a regular or irregular predisposition to opioid substance abuse risk. Such genetic information can be very useful in providing prognostic information about treatment options for a patient. The patient may be associated with a medical condition. The patient may also have already been prescribed a medication for treating the medical condition. The present invention has been found to be advantageous for determining a treatment for a patient who may have a regular or irregular predisposition to opioid substance abuse risk. While the present invention is not necessarily limited to such applications, various aspects of the invention may be appreciated through a discussion of the various examples in this context, as illustrated through the examples below.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims and their equivalents.

Throughout this disclosure, various publications, such as patents and published patent specifications, are referenced by an identifying citation. The disclosures of these publications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)).

### Definitions

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on”, “comprises”, “comprising”, “includes”, “including”, “has”, “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known to those of ordinary skill in the art.

The term “allele” which is used interchangeably herein with the term “allelic variant” refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “haplotype” refers to a combination of alleles on a chromosome or a combination of SNPs within an allele on one chromosome. The alleles or SNPs may or may not be at adjacent locations (loci) on a chromosome. A haplotype may be at one locus, at several loci or an entire chromosome.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure is a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode”, as it is applied to polynucleotides, refers to a polynucleotide which is said to “encode” a polypeptide. The polynucleotide is transcribed to produce mRNA, which is then translated into the polypeptide and/or a fragment thereof by cell machinery. An antisense strand is the complement of such a polynucleotide, and the encoding sequence can be deduced therefrom.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, it includes nucleotide(s) having a uracil base that is uridine.

The terms “oligonucleotide” or “polynucleotide”, or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to part or all of an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, U for Uracil, R for A or G, Y for C, T or U, K for G, T or U, and M for A or C.

As used herein, the terms “drug”, “medication”, and “therapeutic compound” or “compound” are used interchangeably and refer to any chemical entity, pharmaceutical, drug, biological, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound” or “medication” refers to a therapeutic compound that has been shown (e.g., through animal trials or prior experience with administration to humans) to be effective in such treatment. Examples of drugs include, but are not limited to peptides, polypeptides, synthetic organic molecules, naturally occurring organic molecules, nucleic acid molecules, and combinations thereof.

The biological basis for an outcome in a specific patient following a treatment with an opioid pain medication is subject to, inter alia, the patient's genetic predisposition to opioid substance abuse risk. It has been determined that select polymorphisms of a patient, including single nucleotide permutations, haplotypes and phenotypes may be utilized to generate genotype information. The genotype information may be utilized to generate prognostic information. The prognostic information may be utilized in determining treatment options for the patient. The prognostic information is based on the patient's genetic predisposition to opioid substance abuse risk. The prognostic information may also be utilized in determining an expected outcome of a treatment of an individual, such as a treatment with an opioid pain medication.

When a genetic marker such as a polymorphism is used as a basis for determining a treatment for a patient, as described herein, the genetic marker may be measured before or during treatment. The prognostic information obtained may be used by a clinician in assessing any of the following: (a) a probable or likely suitability of an individual to initially receive opioid pain medication treatment(s); (b) a probable or likely unsuitability of an individual to initially receive opioid pain medication treatment(s); (c) a responsiveness to opioid pain medication treatment; (d) a probable or likely suitability of an individual to continue to receive treatment(s); (e) a probable or likely unsuitability of an individual to continue to receive treatment(s); (f) adjusting dosage; (g) predicting likelihood of clinical benefits. As understood by one of skill in the art, measurement of a genetic marker or polymorphism in a clinical setting can be an indication that this parameter may be used as a basis for initiating, continuing, adjusting and/or ceasing administration of opioid pain medication treatment, such as described herein.

Select polymorphisms have been indentified which may be utilized for providing prognostic information, according to the principles of the invention. These findings were correlated with various magnitudes of a positive or negative predispositions to opioid substance abuse risk. Accordingly, assaying the genotype at these markers may be utilized to generate prognostic information which may be utilized to predict the expected outcome of treating the patient with an opioid pain medication based on the expected predisposition of the patient to opioid substance abuse risk. Clinicians prescribing opioid pain medication and other medications may utilize the prognostic information to improve therapeutic decisions and to avoid treatment failures.

Many of the known human single nucleotide permutations (SNPs) are catalogued by the National Center for Biotechnology Information (NCBI) in the Reference SNP (i.e.,“refSNP”) database maintained by NCBI. The Reference SNP database is a polymorphism database (dbSNP) which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

DNA polymorphisms have been identified which may be utilized according to the principles of the invention include SNPs and haplotypes associated with genetic markers in several genes. The genes include the respective genes encoding the Dopamine D1 Receptor (abbreviated DRD1), the Catechol-O-Methyltransferase enzyme (abbreviated COMT), the Dopamine Transporter, also known as Solute Carrier Family 6 Neurotransmitter Transporter, members 3 and 4 (abbreviated respectively as SLC6A3 and SLC6A4), the (GABA)-A Receptor, gamma 2 subunit (abbreviated GABRG2), the Human Kappa Opioid Receptor (abbreviated OPRK1), the Dopamine Beta-Hydroxylase enzyme (abbreviated DBH), the Opioid Receptor, Mu 1 (abbreviated OPRM1), the Dopamine D2 Receptor (abbreviated DRD2), the Methylenetetrahydrofolate Reductase enzyme (abbreviated MTHFR), the Dopamine D4 Receptor (abbreviated DRD4), and the 5-Hydroxytryptamine (Serotonin) Receptor 2A, G Protein-Coupled (abbreviated HTR2A). The DNA polymorphisms which have been identified as active for predicting a genetic predisposition to opioid substance abuse risk are SNP Diploid Polymorphisms. In the identified SNP diploid polymorphisms, the predisposition to opioid substance abuse varies depending upon the active allele of a SNP in a chromosome of a gene as well as the zygosity of the SNP diploid at the locus of the SNP on the chromosome. The SNP diploid polymorphisms identified as predisposition to opioid substance abuse are listed in Table 1 below.

TABLE 1*
Identification of SNP Diploid Polymorphisms

| | | SNP | | | |
|---|---|---|---|---|---|
| | | Diploid | | | |
| No. | rs# | ID** | Zygosity | DNA Context Sequence for Active SNP(s)*** | SEQ ID |
| 1 | rs4532 | DRD1-ANC | homozygous | AGGGGCTCTGACACCCCTCAAGTTCC[T]AAGCAGGGAATAGGGGTCAGTCAGA | SEQ ID No: 1 |
| 2 | rs4532 | DRD1-HET | heterozygous | AGGGGCTCTGACACCCCTCAAGTTCC[C/T]AAGCAGGGAATAGGGGTCAGTCA | SEQ ID No: 2 |
| | | | | GA | |
| 3 | rs4532 | DRD1-NONA | homozygous | AGGGGCTCTGACACCCCTCAAGTTCC[C]AAGCAGGGAATAGGGGTCAGTCAGA | SEQ ID No: 3 |
| 4 | rs4680 | COMT(2)- | homozygous | CCCAGCGGATGGTGGATTTCGCTGGC[G]TGAAGGACAAGGTGTGCATGCCTGA | SEQ ID No: 4 |
| | | ANC | | | |
| 5 | rs4680 | COMT(2)- | heterozygous | CCCAGCGGATGGTGGATTTCGCTGGC[G/A]TGAAGGACAAGGTGTGCATGCCT | SEQ ID No: 5 |
| | | HET | | GA | |
| 6 | rs4680 | COMT(2)- | homozygous | CCCAGCGGATGGTGGATTTCGCTGGC[A]TGAAGGACAAGGTGTGCATGCCTGA | SEQ ID No: 6 |
| | | NONA | | | |
| 7 | rs27072 | SLC6A3- | homozygous | AGTGCCCCTGGGGCAGCCTCAGAGC[C]GGGAGCAGGGAGCAGGGAGGGAGGG | SEQ ID No: 7 |
| | | ANC | | | |
| 8 | rs27072 | SLC6A3- | heterozygous | AGTGCCCCTGGGGCAGCCTCAGAGC[C/T]GGGAGCAGGGAGCAGGGAGGGAGG | SEQ ID No: 8 |
| | | HET | | G | |
| 9 | rs27072 | SLC6A3- | homozygous | AGTGCCCCTGGGGCAGCCTCAGAGC[T]GGGAGCAGGGAGCAGGGAGGGAGGG | SEQ ID No: 9 |
| | | NONA | | | |
| 10 | rs140701 | SLC6A4- | homozygous | CACATAAGGTCTTGTGATGAGAATT[G]TAACTGTTGTTGTGGCTGAGTTTTC | SEQ ID No: 10 |
| | | ANC | | | |
| 11 | rs140701 | SLC6A4- | heterozygous | CACATAAGGTCTTGTGATGAGAATT[A/G]TAACTGTTGTTGTGGCTGAGTTTT | SEQ ID No: 11 |
| | | HET | | C | |
| 12 | rs140701 | SLC6A4- | homozygous | CACATAAGGTCTTGTGATGAGAATT[A]TAACTGTTGTTGTGGCTGAGTTTTC | SEQ ID No: 12 |
| | | NONA | | | |
| 13 | rs211014 | GABRG2- | homozygous | GCAGGCTAAGGCTCAGCAGTTTGGG[C]TCCAAGATGAAAACAGCATGTATGA | SEQ ID No: 13 |
| | | ANC | | | |
| 14 | rs211014 | GABRG2- | heterozygous | GCAGGCTAAGGCTCAGCAGTTTGGG[A/C]TCCAAGATGAAAACAGCATGTATG | SEQ ID No: 14 |
| | | HET | | A | |
| 15 | rs211014 | GABRG2- | homozygous | GCAGGCTAAGGCTCAGCAGTTTGGG[A]TCCAAGATGAAAACAGCATGTATGA | SEQ ID No: 15 |
| | | NONA | | | |
| 16 | rs1051660 | OPRK1- | homozygous | CCGATCCAGATCTTCCGCGGGGAGCC[G]GGCCCTACCTGCGCCCCGAGCGCCT | SEQ ID No: 16 |
| | | ANC | | | |
| 17 | rs1051660 | OPRK1- | heterozygous | CCGATCCAGATCTTCCGCGGGGAGCC[G/A/T]GGCCCTACCTGCGCCCCGAGC | SEQ ID No: 17 |
| | | HET | | GCCT | |
| 18 | rs1051660 | OPRK1- | homozygous | CCGATCCAGATCTTCCGCGGGGAGCC[T]GGCCCTACCTGCGCCCCGAGCGCCT | SEQ ID No: 18 |
| | | NONA | | | |
| 19 | rs1611115 | DBH- | homozygous | AAGGCAGCTGCCCTCAGTCTACTTG[C]GGGAGAGGACAGGAGGGAGAGGTGC | SEQ ID No: 19 |
| | | ANC | | | |
| 20 | rs1611115 | DBH- | heterozygous | AAGGCAGCTGCCCTCAGTCTACTTG[C/T]GGGAGAGGACAGGAGGGAGAGGTG | SEQ ID No: 20 |
| | | HET | | C | |
| 21 | rs1611115 | DBH- | homozygous | AAGGCAGCTGCCCTCAGTCTACTTG[T]GGGAGAGGACAGGAGGGAGAGGTGC | SEQ ID No: 21 |
| | | NONA | | | |
| 22 | rs1799971 | OPRM1- | homozygous | GGTCAACTTGTCCCACTTAGATGGC[A]ACCTGTCCGACCCATGCGGTCCGAA | SEQ ID No: 22 |
| | | ANC | | | |
| 23 | rs1799971 | OPRM1- | heterozygous | GGTCAACTTGTCCCACTTAGATGGC[A/G]ACCTGTCCGACCCATGCGGTCCGA | SEQ ID No: 23 |
| | | HET | | A | |
| 24 | rs1799971 | OPRM1- | homozygous | GGTCAACTTGTCCCACTTAGATGGC[G]ACCTGTCCGACCCATGCGGTCCGAA | SEQ ID No: 24 |
| | | NONA | | | |
| 25 | rs1800497 | DRD2 | homozygous | CTGGACGTCCAGCTGGGCGCCTGCCT[T]GACCAGCACTTTGAGGATGGCTGTG | SEQ ID No: 25 |
| | | (ANKK1)- | | | |
| | | ANC | | | |
| 26 | rs1800497 | DRD2 | heterozygous | CTGGACGTCCAGCTGGGCGCCTGCCT[C/T]GACCAGCACTTTGAGGATGGCTG | SEQ ID No: 26 |
| | | (ANKK1)- | | TG | |
| | | HET | | | |
| 27 | rs1800497 | DRD2 | homozygous | CTGGACGTCCAGCTGGGCGCCTGCCT[C]GACCAGCACTTTGAGGATGGCTGTG | SEQ ID No: 27 |
| | | (ANKK1)- | | | |
| | | NONA | | | |
| 28 | rs1801133 | MTHFR- | homozygous | CTTGAAGGAGAAGGTGTCTGCGGGAG[C]CGATTTCATCATCACGCAGCTTTTC | SEQ ID No: 28 |
| | | ANC | | | |
| 29 | rs1801133 | MTHFR- | heterozygous | CTTGAAGGAGAAGGTGTCTGCGGGAG[C/T]CGATTTCATCATCACGCAGCTTT | SEQ ID No: 29 |
| | | HET | | TC | |
| 30 | rs1801133 | MTHFR- | homozygous | CTTGAAGGAGAAGGTGTCTGCGGGAG[T]CGATTTCATCATCACGCAGCTTTTC | SEQ ID No: 30 |
| | | NONA | | | |
| 31 | rs3758653 | DRD4- | homozygous | CCTCTTTGGTGAAGAGTCCATAGAA[T]TCTCTGCTGCGCTTTGCAAGCACTT | SEQ ID No: 31 |
| | | ANC | | | |
| 32 | rs3758653 | DRD4- | heterozygous | CCTCTTTGGTGAAGAGTCCATAGAA[T/C]TCTCTGCTGCGCTTTGCAAGCACT | SEQ ID No: 32 |
| | | HET | | T | |
| 33 | rs3758653 | DRD4- | homozygous | CCTCTTTGGTGAAGAGTCCATAGAA[C]TCTCTGCTGCGCTTTGCAAGCACTT | SEQ ID No: 33 |
| | | NONA | | | |
| 34 | rs7997012 | HTR2A- | homozygous | TGCCATTATCTTCAAAGACTTAATT[G]ACAATATTTGTCACTTGCCTATGCA | SEQ ID No: 34 |
| | | ANC | | | |
| 35 | rs7997012 | HTR2AHET | heterozygous | TGCCATTATCTTCAAAGACTTAATT[A/G]ACAATATTTGTCACTTGCCTATG | SEQ ID No: 35 |
| | | | | CA | |
| 36 | rs7997012 | HTR2A- | homozygous | TGCCATTATCTTCAAAGACTTAATT[A]ACAATATTTGTCACTTGCCTATGCA | SEQ ID No: 36 |
| | | NONA | | | |

*Unless otherwise indicated, the context sequences are in FASTA format, as presented by NCBI within the rs cluster report identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the ancestral SNP), -HET (heterozygous as including one ancestral and one non-ancestral SNP in the diploid), or -NONA (homozygous for the non-ancestral SNP).
***Brackets (i.e., “[. . .]”) appear within each context sequence to indicate the location (i.e., the “polymorphism marker” or “marker”) of the polymorphic region in the context sequence.

In Table 1, the active polymorphisms are the various diploid pairs of alleles associated with “SNP markers” called “rs numbers” in the refSNP database. Different diploid pairs for each allele have varying activities for generating prognostic information about opioid substance abuse risk. A SNP marker in dbSNP references a SNP cluster report identification number (i.e., the “rs number”) in the refSNP database. The context sequences shown in Table 1 include the allelic variant(s) and the zygosity of the diploid pair identified as active for providing prognostic information according to the principles of the invention. The context sequences include the active polymorphism SNP located in the relevant region of the the gene. The context sequences also include a number of nucleotide bases flanking the active polymorphism SNP in the relevant region of the gene. In the context sequences shown in Table 1, the polymorphic SNP location is shown in brackets within the context sequence for identification purposes. Table 1 also show the rs cluster report number (i.e., the “rs number”) associated with the active polymorphism SNP in dbSNP maintained by NCBI.

Studies have been conducted and it has been determined that SNP diploid polymorphisms identified in Table 1 are predictive of a differential predisposition to opioid substance abuse risk associated with a patient having one or more of SNP diploid polymorphisms. Select SNP diploid polymorphisms in Table 1 are associated with a patient having an elevated opioid substance abuse risk (i.e., predisposed to having a higher risk for opioid dependency or addiction). Other SNP diploid polymorphisms in Table 1 are associated with a patient having a reduced opioid substance abuse risk.

In the studies genetic data were collected via standard Proove Narcotic Risk Test (Revised) consisting of eleven SNP markers in genes affecting neurochemistry in the mesolimbic reward system. Genomic DNA was isolated from buccal swabs obtained from each patient using a proprietary DNA isolation technique and DNA isolation kit (Macherey Nagel GmbH & Co, KG, Germany), according to the manufacturer's instructions. Genotyping was performed using pre-designed TaqMan® assays67 (Applied Biosystems, Foster City, Calif.). Allele-specific fluorescence signals were distinguished by measuring endpoint 6-FAM or VIC fluorescence intensities at 508 nm and 560 nm, respectively, and genotypes were generated using Genotyper® Software V 1.3 (Applied Biosystems, Foster City, Calif.). The DNA Elution Buffer was used as a negative control, and K562 Cell Line DNA (Promega Corporation, Madison, Wis.), was included in each batch of samples tested as positive control. The genetics portion of the data was calculated based on whether subjects were homozygous or heterozygous for the different variants (Table 1). The results of the mathematical analysis on the SNP diploid polymorphisms in Table 1 are listed in Table 2 below.

TABLE 2
Results for SNP Diploid Polymorphisms

| | | | General Impact | Exemplary |
|---|---|---|---|---|
| | | | to Opioid | OD Risk |
| | | SNP Diploid | Dependency | SNP Parameter |
| No. | rs# | ID** | (OD) Risk | Score |
| 1 | rs4532 | DRD1-ANC | Reduced OD Risk | 0 |
| 2 | rs4532 | DRD1-HET | Neutral | 1 |
| 3 | rs4532 | DRD1-NONA | Elevated OD Risk | 2 |
| 4 | rs4680 | COMT(2)-ANC | Reduced OD Risk | 0 |
| 5 | rs4680 | COMT(2)-HET | Neutral | 1 |
| 6 | rs4680 | COMT(2)-NONA | Elevated OD Risk | 2 |
| 7 | rs27072 | SLC6A3-ANC | Elevated OD Risk | 2 |
| 8 | rs27072 | SLC6A3-HET | Neutral | 1 |
| 9 | rs27072 | SLC6A3-NONA | Reduced OD Risk | i) |
| 10 | rs140701 | SLC6A4-ANC | Reduced OD Risk | i) |
| 11 | rs140701 | SLC6A4-HET | Neutral | 1 |
| 12 | rs140701 | SLC6A4-NONA | Elevated OD Risk | 2 |
| 13 | rs211014 | GABRG2-ANC | Reduced OD Risk | 0 |
| 14 | rs211014 | GABRG2-HET | Neutral | 1 |
| 15 | rs211014 | GABRG2-NONA | Elevated OD Risk | 2 |
| 16 | rs1051660 | OPRK1-ANC | Reduced OD Risk | 0 |
| 17 | rs1051660 | OPRK1-HET | Neutral | 1 |
| 18 | rs1051660 | OPRK1-NONA | Elevated OD Risk | 2 |
| 19 | rs1611115 | DBH-ANC | Reduced OD Risk | 0 |
| 20 | rs1611115 | DBH-HET | Neutral | 1 |
| 21 | rs1611115 | DBH-NONA | Elevated OD Risk | 2 |
| 22 | rs1799971 | OPRM1-ANC | Elevated OD Risk | 2 |
| 23 | rs1799971 | OPRM1-HET | Neutral | 1 |
| 24 | rs1799971 | OPRM1-NONA | Reduced OD Risk | 0 |
| 25 | rs1800497 | DRD2(ANKK1)- | Reduced OD Risk | 0 |
| | | ANC | | |
| 26 | rs1800497 | DRD2(ANKK1)- | Neutral | 1 |
| | | HET | | |
| 27 | rs1800497 | DRD2(ANKK1)- | Elevated OD Risk | 2 |
| | | NONA | | |
| 28 | rs1801133 | MTHFR-ANC | Reduced OD Risk | 0 |
| 29 | rs1801133 | MTHFR-HET | Neutral | 1 |
| 30 | rs1801133 | MTHFR-NONA | Elevated OD Risk | 2 |
| 31 | rs3758653 | DRD4-ANC | Reduced OD Risk | 0 |
| 32 | rs3758653 | DRD4-HET | Neutral | 1 |
| 33 | rs3758653 | DRD4-NONA | Elevated OD Risk | 2 |
| 34 | rs7997012 | HTR2A-ANC | Reduced OD Risk | 0 |
| 35 | rs7997012 | HTR2AHET | Neutral | 1 |
| 36 | rs7997012 | HTR2A-NONA | Elevated OD Risk | 2 |

*Reference Nos. and IDs in Table 2 coincide with same Reference Nos. and IDs appearing in Table 1.

Several demographic phenotypes of the subjects in the studies were also analyzed to determine correlations with higher or lower opioid dependency risk. Phenotypic data were collected via ORT and SOAPP®-R.

The ORT is a 5-question office-based survey, which can be completed by the patient or their physician. The questionnaire assesses potential opioid risk factors such as personal/family history of alcohol, prescription drug, or illegal drug abuse; history of childhood sexual abuse; and the presence of psychiatric disorders. It can be utilized to stratify opioid abuse risk or opioid dependency risk into different levels, such as for example, three levels: low (scores 0-3), moderate (scores 4-7), and high (scores ≥8).

The SOAPP®-R survey contains empirically-derived 24 items that have been empirically found to identify aberrant medication-related behaviors. It is not intended for stratifying the risk of opioid abuse. Rather, the SOAPP®-R outcome provides physicians with the information about the level of monitoring required for chronic pain patients considered for long-term opioid therapy. Questions focus, for example, on how often a patient feels bored, overwhelmed, or impatient with their doctor; how often a patient has been in certain situations (such as being arrested, undergoing treatment for addiction, or having been told by others they may have a substance abuse problem), and if the patient has engaged in aberrant medication-related behaviors such as counting pills, taking more than a prescribed amount of medication, and borrowing medicine from another person. Each item has a choice of 5 responses ranging from “never” to “very often”.

Six phenotypic characteristics were selected from the ORT. These phenotypes included a patient characterized as having (1) an age of 16-45 years, (2) a personal history of alcoholism or alcohol abuse, (3) a personal history of illegal drug abuse, (4) a personal history of prescription drug abuse, (5) a personal history of depression, and/or (6) a personal history of other mental health diseases or disorders including attention deficit disorder, obsessive compulsive disorder, bipolar disorder, and schizophrenia. Depression was assessed and scored independently from the other mental health disorders because of its particularly strong association with opioid abuse. A polynomial regression analysis was performed and statistical significance was found among the different demographic phenotypes to objectively stratify the Opioid Dependency (OD) Risk associated with the various phenotypes. The results of the mathematical analysis on the phenotypes are listed in Table 3 below.

TABLE 3
Results for Phenotype Polymorphisms

| | | General Impact to | Exemplary OD Risk |
|---|---|---|---|
| | | Opioid Dependency | Phenotype |
| No. | Phenotype Identification | (OD) Risk | Parameter Score |
| 1 | Age between 16 and 45 years | Elevated OD Risk | 4 |
| 2 | Personal history of a mental health disorder | Elevated OD Risk | 4 |
| 3 | Depression | Elevated OD Risk | 6 |
| 4 | Personal history of alcoholism | Elevated OD Risk | 4 |
| 5 | Personal history of illegal drug use | Elevated OD Risk | 6 |
| 6 | Personal history of prescription (legal) drug abuse | Elevated OD Risk | 6 |

The OD Risk Phenotype Parameter scores from Table 3 and the OD Risk SNP Parameter Scores from Table 2 may be added to form a Total OD Risk score. The Total OD Risk score may be compared with threshhold levels for ranking the total level of OD Risk associated with a patient. For example, according to an embodiment, a Total OD Risk score for a patient may range in a predetermined scale, such as, for example, from 0 to 52 for the twelve (12) genetic phenotypes shown in Tables 1 and 2 and the six (6) demographic phenotypes shown in Table 3. According to an embodiment, a threshold of thirteen (13) as the lower limit of a Total OD Risk score may indicate an elevated OD risk. According to another embodiment, a Total OD Risk score range may be stratified, such as 0-11 as representing a range for low OD Risk, 12-23 as a range for moderate OD Risk and 24-52 as a range for high OD Risk.

The invention further provides systems and methods which utilize one or more determinations of the presence and/or absence of one or more of the polymorphisms listed in Tables 1 through 3. For example, information obtained using the diagnostic assays described herein is useful for determining a potential OD risk in a patient and a likely response if administered an opioid medication and/or a likelihood of a positive response to the treatment. Based on this prognostic information, a clinician can recommend a therapeutic protocol useful for treating an individual based on their genetic predisposition to OD risk or adjust a previously administered therapy to accommodate the patient's OD risk.

For example, a method provided by the invention is a diagnostic method for determining the OD risk associated with a patient which method is not practiced on the patient's body, i.e. is an ex vivo diagnostic method. The method may involve determining patient information which may be obtained by assaying a sample of genetic material associated with the patient. The method does not involve obtaining the sample from the patient's body. The invention also provides uses of the systems and methods, for example of the diagnostic assays, for determining the OD risk associated with a patient.

In addition, knowledge of the identity of a particular polymorphism in an individual's genetic profile allows customization of medication or therapy based on the particular individual's genetic profile. For example, an individual's genetic profile can enable a doctor to more effectively prescribe a drug that will address the patient's medical condition or to better determine an appropriate dosage of a particular drug.

Detection of point mutations or other types of the allelic variants In Tables 1 and 2 may be accomplished several ways known in the art, such as by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

One such detection method is allele specific hybridization using probes overlapping the polymorphic region and having, for example, about 5, or alternatively 10, or alternatively 20, or alternatively 25, or alternatively 30 nucleotides around the polymorphic region. In another embodiment, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

If the polymorphic region is located in the coding region of the gene of interest, yet other methods than those described above can be used for determining the identity of the allelic variant according to methods known in the art.

The genotype information obtained from analyzing a sample of a patient's genetic material may be utilized, according to the principles of the invention, to predict whether a patient has a level of risk associated with opioid dependency. The risk may be associated with a side effect the patient may be susceptible to developing, an efficacy of the drug to the patient specifically or some combination thereof. The genotype information of the patient may be combined with demographic information about the patient as described above.

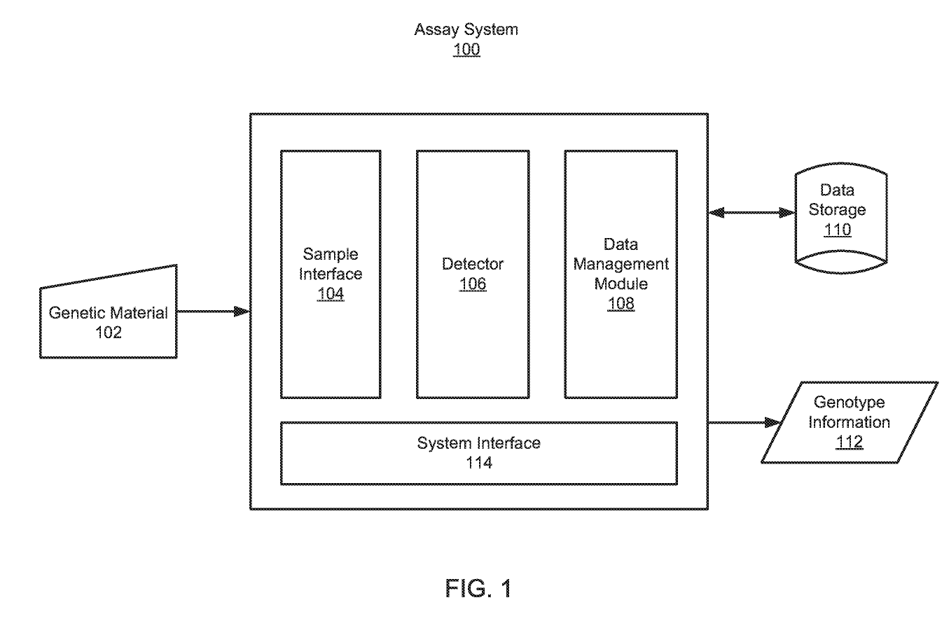

Referring to FIG. 1, depicted is an assay system 100. An assay system, such as assay system 100, may access or receive a genetic material, such as genetic material 102. The sample of genetic material 102 can be obtained from a patient by any suitable manner. The sample may be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Table I above. The genotype information 112 may be stored in data storage 110 or transmitted to another system or entity via a system interface 114.

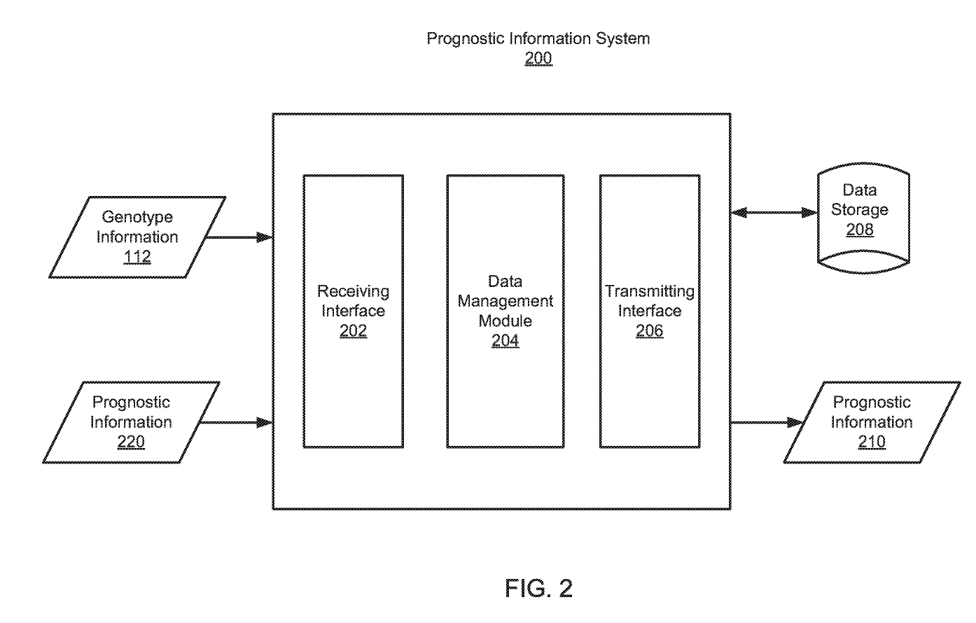

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

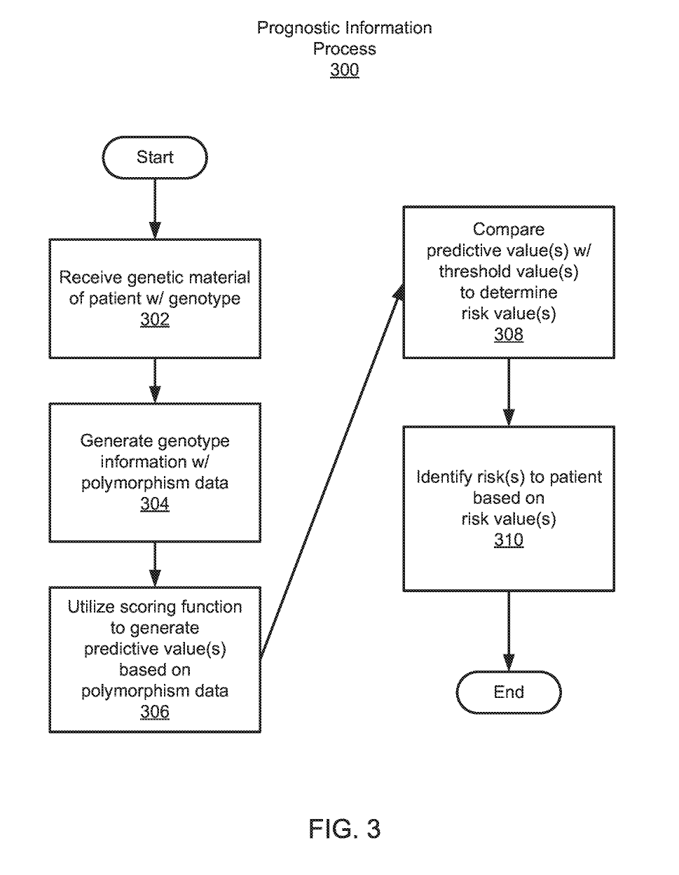

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Tables 1 and 2. The demographic phenotype information described in Table 3 may alternatively be obtained through input of information provided by the patient in an input survey. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign predictive values and aggregate values.

For example, an additive effect algorithm may be utilized to generate an analysis of a patient's genetic predisposition and their demographic phenotype predisposition to opioid dependency risk. In the additive effect algorithm, polymorphism data of the genotype information obtained from analyzing a patient's genetic material is utilized to indicate the active polymorphisms identified from a patient's genotype information. A tested polymorphism may be determined to be (1) absent or present in either (2) a heterozygous or (3) a homozygous variant in the patient's genotype. According to the additive effect algorithm, the polymorphisms identified from a patient's genotype information and demographic phenotype are each assigned a real value, such as an Odds Ratio (OR) or a parameter score, depending on which polymorphisms appears in the patient's genotype and demographic information.

In an illustrative example, if the DRD1-NONA genetic polymorphism (i.e., the Non-Ancestral variant of the Dopamine D1 Receptor (abbreviated DRD1) associated with cluster report no. rs4532—See No. 3 in Tables 1 and 2) appears in the genotype information generated based on a DNA sample, an exemplary value of +2 (the OD Risk SNP Parameter Score associated with DRD1-ANC in Table 1) may be assigned as a predictive value associated with the DRD1-NONA diploid polymorphism. The scoring function associated with the predictive value of the OD Risk SNP Parameter Score is the result of a multinomial logistic regression analysis performed using SPSS to generate the OD Risk associated with DRD1-NONA in Table 1. Other scoring functions may also be utilized as long as the predictive value generated reflects an elevated Opioid Dependency Risk associated with the DRD1-NONA diploid polymorphism.

In another example, genetic polymorphisms DRD1-ANC (OD=0), COMT(2)-HET (OD=1), SLC6A3-HET (OD=1), SLC6A4-ANC (OD=0), GABRG2-NONA (OD=2), OPRK1-NONA (OD=2), DBH-NONA (OD=2), OPRM1-HET (OD=1), DRD2(ANKK1)-NONA (OD=2), MTHFR-HET (OD=1), DRD4- NONA (OD=0) and HTR2A-ANC (OD=0) appear in the genotype information generated based on a DNA sample. The aggregate value of the diploid polymorphism results in this example totals +12 [i.e., 0+1+1+0+2+2+2+1+2+1+0+0]. At the same time, demographic data determined for the the patient demonstrates that the patient's age is 22 (OD=4), suffers from depression (OD=6) and has a history of alcoholism (OD=4), but has none of the other demographic phenotype characteristics listed in Table 3. Thus the demographic phenotype total is 14 (4+6+4), based on Table 3, although other numbers may be applied if derived from different polynomial analyses. In this example, a total OD Risk score for the patient is 26 (12+14) (i.e., a parameter score of 12 associated with genetic information and a parameter score of 14 associated with the demographic phenotypes).

To gather data for the algorithm, one or more of the SNP Diploid Polymorphisms, such as those listed in Table 1, may be tested and/or analyzed to produce one or more values associated with the presence or absence of the SNP Diploid Polymorphisms. In another example, one or more of the characteristic phenotypes in Table 3 may be tested and/or analyzed to produce one or more values associated with the presence or absence of the demographic phenotypes in Table 3. Other factors, such as other SNP Diploid Polymorphisms, other demographic phenotypes may also be tested and/or analyzed to produce one or more values associated with the presence or absence of the other SNP Diploid Polymorphisms and other demographic phenotypes.

The values gathered are based on results of the various tests and data gathered and/or determined. The values may be factored into an algorithm to score a subject's opioid dependency risk based on the subject's genetic information and/or non-genetic characteristics or phenotypes. The algorithm may compute a composite score based on the results of individual tests. The composite score may be calculated based on an additive analysis of the individual scores which may be compared with a threshold value for determining OD risk based on the additive score. In addition or in the alternative, more complex functions may be utilized to process the values developed from the testing results, such as utilizing one or more weighting factor(s) applied to one or more of the individual values based on various circumstances, such as if a subject was tested using specific equipment, a temporal condition, etc.

In all of the preceding examples, the predictive values and aggregate values generated are forms of prognostic information 210.

At step 310, the result of the comparison obtained in step 308 generates a second form of prognostic information 220. For example, (a) if the determined sum is higher than the threshold value, it can be predicted that the patient is at an elevated risk for opioid dependency associated with prescribing the patient an opioid medication; (b) if the determined sum is at or near the threshold value, it can be predicted that the patient is at a moderate risk for for opioid dependency; and (c) if the determined sum is below the threshold value, it can be predicted that the patient is at a low risk for opioid dependency.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300. Other algorithms may also be used in a similar manner to generate useful forms of prognostic information for determining treatment options for a patient.

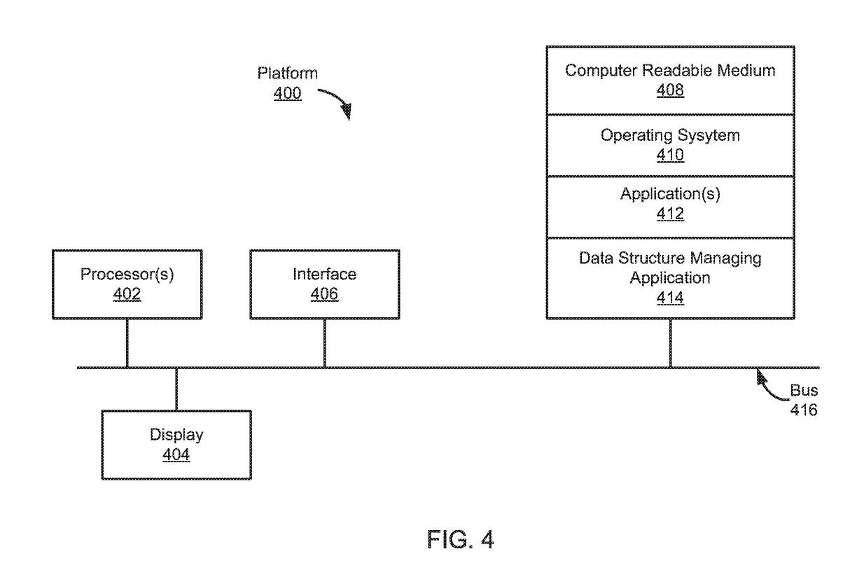

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

What is claimed is:

1. A method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group consisting of
DRD1-ANC, DRD1-HET, and DRD1-NONA in the DRD1 gene,
COMT(2)-ANC, COMT(2)-HET, and COMT(2)-NONA in the COMT gene,
SLC6A3-ANC, SLC6A3-HET, and SLC6A3-NONA in the SLC6A3 gene,
SLC6A4-ANC, SLC6A4-HET, and SLC6A4-NONA in the SLC6A4 gene,
GABRG2-ANC, GABRG2-HET, and GABRG2-NONA in the GABRG2 gene,
OPRK1-ANC, OPRK1-HET, and OPRK1-NONA in the OPRK1 gene,
DBH-ANC, DBH-HET, and DBH-NONA in the DBH gene,
OPRM1-ANC, OPRM1-HET, and OPRM1-NONA in the OPRM1 gene,
DRD2(ANKK1)-ANC, DRD2(ANKK1)-HET, and DRD2(ANKK1)-NONA in the DRD2 gene,
MTHFR-ANC, MTHFR-HET, and MTHFR-NONA in the MTHFR gene,
DRD4-ANC, DRD4-HET, and DRD4-NONA in the DRD4 gene, and
HTR2A-ANC, HTR2A-HET, and HTR2A-NONA in the HTR2A gene; and
determining an opioid dependency risk associated with the human subject based, at least in part, on the presence or absence of the one or more SNP diploid polymorphisms in the subject genotype.

2. A method of claim 1, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype,
wherein characteristics of the corresponding region of the reference genotype are based upon a predetermined population norm;
determining prognostic information associated with the human subject based on the determined opioid dependency risk; and
determining a therapy for the human subject based on the determined prognostic information associated with the human subject,
wherein the method for determining the opioid dependency risk associated with the human subject, is an ex vivo method.

3. A method of claim 1, wherein the (1) data and/or (2) information and/or (3) at least one signal are further based, at least in part, on the following:
determining demographic information associated with the human subject as part of the patient information; and
determining from the demographic information whether the human subject is characterized as being associated with one or more demographic phenotypes,
wherein the determining of the opioid dependency risk associated with the human subject is based, at least in part, on the presence or absence of the one or more demographic phenotypes in the patient information associated with the human subject.

4. A method of claim 3,
wherein the one or more demographic phenotypes associated with the human subject are selected from
a factor associated with the age of the human subject,
a history of a mental health disorder other than depression in the human subject,
a history of depression in the human subject,
a history of alcoholism in the human subject,
a history of illegal drug use by the human subject,
a history of legal drug abuse by the human subject, or
a combination thereof.

5. A method of claim 1, wherein the one or more SNP diploid polymorphisms include at least two SNP diploid polymorphisms from the SNP diploid group.

6. A method of claim 1, wherein the one or more SNP diploid polymorphisms include at least four SNP diploid polymorphisms from the SNP diploid group.

7. A method of claim 1, wherein the one or more SNP diploid polymorphisms include at least eight SNP diploid polymorphisms from the SNP diploid group.

8. A method of claim 1, wherein the one or more SNP diploid polymorphisms include at least twelve SNP diploid polymorphisms from the SNP diploid group.

9. An apparatus comprising:
at least one processor; and
at least one memory including computer program code for one or more programs,
the at least one memory and the computer program code configured to, with the at least one processor, cause the apparatus to perform at least the following,
determine patient information, including DNA information, associated with a human subject;
determine from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group consisting of
DRD1-ANC, DRD1-HET, and DRD1-NONA in the DRD1 gene,
COMT(2)-ANC, COMT(2)-HET, and COMT(2)-NONA in the COMT gene,
SLC6A3-ANC, SLC6A3-HET, and SLC6A3-NONA in the SLC6A3 gene,
SLC6A4-ANC, SLC6A4-HET, and SLC6A4-NONA in the SLC6A4 gene,
GABRG2-ANC, GABRG2-HET, and GABRG2-NONA in the GABRG2 gene,
OPRK1-ANC, OPRK1-HET, and OPRK1-NONA in the OPRK1 gene,
DBH-ANC, DBH-HET, and DBH-NONA in the DBH gene,
OPRM1-ANC, OPRM1-HET, and OPRM1-NONA in the OPRM1 gene,
DRD2(ANKK1)-ANC, DRD2(ANKK1)-HET, and DRD2(ANKK1)-NONA in the DRD2 gene,
MTHFR-ANC, MTHFR-HET, and MTHFR-NONA in the MTHFR gene,
DRD4-ANC, DRD4-HET, and DRD4-NONA in the DRD4 gene, and
HTR2A-ANC, HTR2A-HET, and HTR2A-NONA in the HTR2A gene; and
determine an opioid dependency risk associated with the human subject based, at least in part, on the presence or absence of the one or more SNP diploid polymorphisms in the subject genotype.

10. An apparatus of claim 9, wherein the apparatus is further caused to:
determine a comparing of a region, including the one or more SNP diploid polymorphisms, of the subject genotype with a corresponding region of a predetermined reference genotype,
wherein characteristics of the corresponding region of the reference genotype are based upon a predetermined population norm;
determine prognostic information associated with the human subject based on the determined opioid dependency risk; and
determine a therapy for the human subject based on the determined prognostic information associated with the human subject,
wherein the methodology for determining the opioid dependency risk associated with the human subject associated with the apparatus, is an ex vivo methodology.

11. An apparatus of claim 9, wherein the apparatus is further caused to:
determine demographic information associated with the human subject as part of the patient information; and
determine from the demographic information whether the human subject is characterized as being associated with one or more demographic phenotypes,
wherein the determination of the opioid dependency risk associated with the human subject is based, at least in part, on the presence or absence of the one or more demographic phenotypes in the patient information associated with the human subject.

12. An apparatus of claim 11,
wherein the one or more demographic phenotypes associated with the human subject are selected from
a factor associated with the age of the human subject,
a history of a mental health disorder other than depression in the human subject,
a history of depression in the human subject,
a history of alcoholism in the human subject,
a history of illegal drug use by the human subject,
a history of legal drug abuse by the human subject, or
a combination thereof.

13. An apparatus of claim 9, wherein the one or more SNP diploid polymorphisms include at least two SNP diploid polymorphisms from the SNP diploid group.

14. An apparatus of claim 9, wherein the one or more SNP diploid polymorphisms include at least four SNP diploid polymorphisms from the SNP diploid group.

15. A non-transitory computer readable medium storing computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group consisting of
DRD1-ANC, DRD1-HET, and DRD1-NONA in the DRD1 gene,
COMT(2)-ANC, COMT(2)-HET, and COMT(2)-NONA in the COMT gene,
SLC6A3-ANC, SLC6A3-HET, and SLC6A3-NONA in the SLC6A3 gene,
SLC6A4-ANC, SLC6A4-HET, and SLC6A4-NONA in the SLC6A4 gene,
GABRG2-ANC, GABRG2-HET, and GABRG2-NONA in the GABRG2 gene,
OPRK1-ANC, OPRK1-HET, and OPRK1-NONA in the OPRK1 gene,
DBH-ANC, DBH-HET, and DBH-NONA in the DBH gene,
OPRM1-ANC, OPRM1-HET, and OPRM1-NONA in the OPRM1 gene,
DRD2(ANKK1)-ANC, DRD2(ANKK1)-HET, and DRD2(ANKK1)-NONA in the DRD2 gene,
MTHFR-ANC, MTHFR-HET, and MTHFR-NONA in the MTHFR gene,
DRD4-ANC, DRD4-HET, and DRD4-NONA in the DRD4 gene, and
HTR2A-ANC, HTR2A-HET, and HTR2A-NONA in the HTR2A gene; and
determining an opioid dependency risk associated with the human subject based, at least in part, on the presence or absence of the one or more SNP diploid polymorphisms in the subject genotype.

16. A computer readable medium of claim 15, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype,
wherein characteristics of the corresponding region of the reference genotype are based upon a predetermined population norm;
determining prognostic information associated with the human subject based on the determined opioid dependency risk; and
determining a therapy for the human subject based on the determined prognostic information associated with the human subject
wherein the methodology for determining the opioid dependency risk associated with the human subject associated with the computer readable medium, is an ex vivo methodology.

17. A computer readable medium of claim 15, wherein the (1) data and/or (2) information and/or (3) at least one signal are further based, at least in part, on the following:
determining demographic information associated with the human subject as part of the patient information; and
determining from the demographic information whether the human subject is characterized as being associated with one or more demographic phenotypes,
wherein the determining of the opioid dependency risk associated with the human subject is based, at least in part, on the presence or absence of the one or more demographic phenotypes in the patient information associated with the human subject.

18. A computer readable medium of claim 17,
wherein the one or more demographic phenotypes associated with the human subject are selected from
a factor associated with the age of the human subject,
a history of a mental health disorder other than depression in the human subject,
a history of depression in the human subject,
a history of alcoholism in the human subject,
a history of illegal drug use by the human subject,
a history of legal drug abuse by the human subject, or
a combination thereof.

19. A computer readable medium of claim 15, wherein the one or more SNP diploid polymorphisms include at least two SNP diploid polymorphisms from the SNP diploid group.

20. A computer readable medium of claim 15, wherein the one or more SNP diploid polymorphisms include at least four SNP diploid polymorphisms from the SNP diploid group.
